(19) United States
(12) Patent Application Publication
(10) Pub. No.: US 2025/0045982 A1
(43) Pub. Date: Feb. 6, 2025

(54) METHODS AND RELATED ASPECTS FOR MITIGATING UNKNOWN BIASES IN COMPUTED TOMOGRAPHY DATA

(71) Applicant: THE JOHNS HOPKINS UNIVERSITY, Baltimore, MD (US)

(72) Inventors: Joseph Webster STAYMAN, Baltimore, MD (US); Jianan GANG, Baltimore, MD (US)

(21) Appl. No.: 18/715,998

(22) PCT Filed: Dec. 14, 2022
(86) PCT No.: PCT/US2022/052789

§ 371 Date: Jun. 3, 2024

Related U.S. Application Data
(60) Provisional application No. 63/289,708, filed on Dec. 15, 2021.

(51) Int. Cl. G06T 11/00

(52) U.S. Cl. CPC G06T 11/005; G06T 11/008; G06T 2211/408; G06T 2211/441

(57) ABSTRACT

Provided herein are methods of reconstructing computed tomography (CT) images and methods of generating artificial neural networks (ANNs) to estimate unmodeled bias from acquired CT projection data. Related systems and computer program products are also provided.

### CROSS-REFERENCE TO RELATED APPLICATIONS

This application is the national stage entry of International Patent Application No. PCT/US2022/052789, filed on Dec. 14, 2022, and published as WO 2023/114265 A1 on Jun. 22, 2023, which claims the benefit of U.S. Provisional Patent Application Ser. No. 63/289,708, filed on Dec. 15, 2021, which are hereby incorporated by reference in their entireties.

### STATEMENT REGARDING FEDERALLY SPONSORED RESEARCH OR DEVELOPMENT

This invention was made with government support under grant R21EB026849 awarded by the National Institutes of Health. The government has certain rights in the invention.

### BACKGROUND

Computed tomography (CT) remains a valuable technology across a range of diagnostics and interventional imaging situations. New CT systems continue to be developed using new technologies including novel sources, detectors, and system geometries. Both traditional and novel systems are subject to various physical effects that make the actual data acquisition deviate from the idealized reconstruction model. The potential sources of such modeling errors are widespread and can include inexact calibrations of the x-rays produced by the source, the filtration of those x-rays, physical effects induced by the patient (e.g. beam hardening and scatter), and detector effects. While a multitude of approaches can be used to mitigate such errors, there are often residual biases present—e.g. inexact knowledge of x-ray spectrum for beam hardening correction, drift in detectors, tube warm-up effects, inexact modeling of scatter due to a limited number of photon tracked and simulated via Monte Carlo, etc. Any of these unmodeled biases has the potential to propagate through reconstruction to create artifacts and errors in the estimation of attenuation coefficients. Such degradations have the potential to impact clinical diagnoses and can confound quantitative imaging that relies on accurate estimation of attenuation (e.g. for attenuation correction in PET/CT and in radiation therapy treatment planning [Schneider et al., “The calibration of CT hounsfield units for radiotherapy treatment planning.” Phys Med Biol. 1996; 41:111]).

While there has been a great deal of effort to address individual biases through specific modeling and correction efforts, it has proven difficult to find a solution that covers all sources of bias. Therefore, there remains a need to address these unknown or “impossible” to model biases based, for example, only on the acquired projection data. A significant amount of research has gone into sinogram consistency critera [Lesaint, “Data consistency conditions in X-ray transmission imaging and their application to the self-calibration problem,” Functional Analysis [math.FA]. Communauté Universite Grenoble Alpes, 2018. English.] in which specific properties of legitimate sinograms are expressed mathematically. For example, the integral of the logarithm of projections is constant in each slice of parallel projection data. More general consistency criteria apply, but they are more complicated mathematical expressions and may not hold under all circumstances (e.g. truncated data). However, the existence of such criteria means that not all sinograms are possible and there is a potential to identify and correct the sinograms to be consistent.

Accordingly, there is a need for additional methods, and related aspects, for reconstructing CT images that mitigates biases, particularly unknown or unmodeled biases.

### SUMMARY

The present disclosure relates, in certain aspects, to methods of reconstructing computed tomography (CT) images that mitigate unmodeled biases. In some aspects, the present disclosure also provides methods of generating artificial neural networks (ANNs) to estimate unmodeled bias from acquired CT projection data. Related systems and computer readable media are also provided.

In one aspect, for example, the present disclosure provides a method of reconstructing a CT image. The method includes receiving acquired CT projection data of an object (e.g., a subject or the like). The method also includes substantially removing at least one unmodeled bias from the acquired CT projection data using at least one trained artificial neural network (ANN) and at least one loss function that incorporates intermediate CT reconstruction information to produce corrected CT projection data. In addition, the method also includes generating a reconstructed CT image from the corrected CT projection data, thereby reconstructing the CT image.

In another aspect, the present disclosure provides a method of generating an artificial neural network (ANN) to estimate unmodeled bias from acquired computed tomography (CT) projection data. The method includes training at least one ANN comprising at least one loss function that incorporates intermediate CT reconstruction information to estimate unbiased projections from a plurality of biased and/or unbiased sinogram pairs, thereby generating an ANN to estimate unmodeled bias from acquired CT projection data.

In another aspect, the present disclosure provides a computed tomography (CT) system that includes at least one x-ray energy source and at least one x-ray detector configured and positioned to detect x-ray energy transmitted through an object (e.g., a subject or the like) from the x-ray energy source. The system also includes at least one trained artificial neural network (ANN) and at least one loss function that incorporates intermediate CT reconstruction information that are configured to remove unmodeled bias from acquired CT projection data. In addition, the system also includes at least one controller that is operably connected, or connectable, at least to the x-ray detector and to the trained ANN. The controller comprises, or is capable of accessing, computer readable media comprising non-transitory computer executable instructions which, when executed by at least one electronic processor, perform at least: receiving acquired CT projection data of an object; substantially removing at least one unmodeled bias from the acquired CT projection data using the trained ANN and the loss function to produce corrected CT projection data; and, generating a reconstructed CT image from the corrected CT projection data.

In another aspect, the present disclosure provides a computer readable media comprising non-transitory computer executable instruction which, when executed by at least electronic processor, perform at least: receiving acquired CT projection data of an object; substantially removing at least one unmodeled bias from the acquired CT projection data using at least one trained artificial neural network (ANN) and at least one loss function that incorporates intermediate CT reconstruction information to produce corrected CT projection data; and generating a reconstructed CT image from the corrected CT projection data.

In some embodiments, a source of the unmodeled bias is unknown or substantially indeterminable. In some embodiments, the methods include (or the instructions of the system or computer readable media further perform at least) using at least one physical model of a CT data collection process to identify the unmodeled bias from the acquired CT projection data. In some embodiments, the methods and other aspects disclosed herein include using at least one physical model of a CT data collection process, and/or CT data collected from physical calibration phantoms (e.g., existing or custom designed phantoms) to identify the unmodeled bias from the acquired CT projection data.

In some embodiments, the unmodeled bias comprises a potential to propagate through a reconstruction process to create one or more artifacts and/or one or more errors in an estimation of attenuation coefficients. In some embodiments, the unmodeled bias is selected from the group consisting of, for example, a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

In some embodiments, the unmodeled bias comprises at least one residual bias. In some embodiments, the residual bias is selected from the group consisting of, for example, an inexact knowledge of an x-ray spectrum for beam hardening correction, a drift in one or more x-ray detectors, a tube warm-up effect, and an inexact modeling of scatter. In some embodiments, the unmodeled bias is one-dimensional (1D). In some of these embodiments, the 1D unmodeled bias is a function of projection angle. In some embodiments, the unmodeled bias is two-dimensional (2D). In some of these embodiments, the 2D unmodeled bias is a function of radial detector bin and projection angle. In some embodiments, the unmodeled bias is substantially specific to an anatomy of the object. In some embodiments, the unmodeled bias comprises scatter and/or differential beam hardening.

In some embodiments, the loss function comprises a spatial-frequency loss function, an image domain loss function, and/or a projection domain loss function. In some embodiments, the loss function comprises a mean squared error between ramp-filtered ANN-corrected and unbiased projection data in a frequency domain. In some embodiments, the spatial-frequency loss function comprises the Formula:

$$Loss = \frac{1}{N} {\sum }_{i} {\sum }_{j} {\{ F [ Ramp filter * {y}_{CNN corrected} ( i , j ) ] - F [ Ramp filter * {y}_{unbiased} ( i , j ) ] \}}^{2}$$

where N is a number of measurements, F{ }represents a Fourier transform, * represents convolution, y_{CNN corrected} represents de-biased projections, y_{unbiased} are true projections, and (i,j) are measurement indices.

In some embodiments, the acquired CT projection data of the object is generated using a single energy CT (SECT) technique. In some embodiments, acquired CT projection data of the object is generated using a spectral CT technique. In some of these embodiments, for example, the spectral CT technique comprises a dual energy CT (DECT) technique and/or a photon counting CT technique.

In some embodiments, the ANN is trained to estimate unbiased projections from a plurality of biased and/or unbiased sinogram pairs. In some embodiments, the plurality of biased and/or unbiased sinogram pairs comprises about 10,000, 20,000, 30,000, 40,000, 50,000, 60,000, 70,000, 80,000, 90,000, 100,000, or more biased and/or unbiased sinogram pairs. In some embodiments, the ANN is not trained to estimate a specific bias type. In some embodiments, the ANN comprises a convolutional neural network (CNN) that comprises at least one encoding phase, at least one bridge, at least one decoding phase, and at least one two-dimensional (2D) convolutional layer. In some of these embodiments, the 2D convolutional layer is configured to learn a one-dimensional (1D) bias map. In some of these embodiments, the 2D convolutional layer is configured to learn a 2D bias map and/or a 3D bias map.

In some embodiments, the corrected CT projection data is improved in a projection domain, an image domain, or both a projection domain and an image domain relative to the acquired CT projection data. In some embodiments, the methods include (or the instructions of the system or computer readable media further perform at least) using a reconstruction technique selected from the group consisting of: a model-based iterative reconstruction (MBIR) technique, a deep learning technique, and a filtered-backprojection (FBP) technique. In some embodiments, the methods include receiving the x-ray CT data at one or more x-ray detectors configured and positioned to detect x-ray energy transmitted through the object from one or more x-ray energy sources to generate the acquired CT projection data of the object.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps of reconstructing a computed tomography (CT) image according to some aspects disclosed herein.

FIG. 2 is a flow chart that schematically depicts exemplary method steps of generating an artificial neural network (ANN) to estimate unmodeled bias from acquired computed tomography (CT) projection data according to some aspects disclosed herein.

FIG. 3 is a schematic diagram of an exemplary system suitable for use with certain aspects disclosed herein.

FIG. 4 is a plot showing an illustration of projection-dependent tube warm up scaling factors applied to the projection data after mean-I₀-correction.

FIG. 5 is an image showing a sample random 2-D bias using Gaussian blobs.

FIG. 6 (panels a-d) show a) FBP and b) PL reconstructions of unbiased- and tube-warm-up-biased-projections and difference. c) FBP and d) PL reconstructions of unbiased- and 2D-biased projections and difference. All reconstructions are leveled at the mean HU of the region-of-interest (ROI) and displayed with window+/−150 HU.

FIG. 7 (panels a and b) schematically show a) ResUNet structure for CT-debiasing and b) details of the residual block.

FIG. 8 (panels a and b) show training history of the CNN as well as the spatial-frequency loss comparison in both training and test datasets for a) the 1D unmodeled tube-warmup scenario, and b) the 2D generalized bias scenario.

FIG. 9 (panels a and b) show a) unbiased, mean-I₀-corrected and CNN corrected projections and b) show difference maps with respect to the unbiased projection. (All projections are shown as post-log line integrals).

FIG. 10 (panels a and b) show a) FBP and b) PL reconstructions from unbiased, mean-I₀-corrected and CNN-corrected projections and the associated difference maps. All reconstructions are leveled at the mean HU of the ROI and displayed with window+/−150 HU.

FIG. 11 (panels a and b) show a) unbiased, 2D biased and CNN-corrected projections and b) show difference maps with respect to the unbiased projection. (All projections are shown aspost-log line integrals).

FIG. 12 (panels a and b) show a) FBP and b) PL reconstructions from unbiased, 2D biased and CNN-corrected projections, and the associated difference maps. All reconstructions are leveled at the mean HU of the ROI and displayed with window+/−150 HU.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

Machine Learning Algorithm: As used herein, “machine learning algorithm,” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

Substantially: As used herein, “substantially,” “about,” or “approximately” as applied to one or more values or elements of interest, refers to a value or element that is similar to a stated reference value or element. In certain embodiments, the term “substantially,” “about, or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Unmodeled Bias: As used herein, “unmodeled bias” in the context of computed tomography (CT) image reconstruction, refers to mismatches or discrepancies between a physical model of CT data collection process and a corresponding actual system under consideration.

### DETAILED DESCRIPTION

Proper reconstruction of computed tomography (CT) image volumes typically utilizes a physical model of the data collection process. Any mismatches between the physical model and the actual system represent unmodeled biases. Such unmodeled biases can arise from a multitude of sources including varying system “drifts” in the source and detector, as well as unmodeled physics (like incomplete scatter rejection or inexact scatter correction). Generally, these biases introduce artifacts and quantitation errors that degrade image quality and make quantitative data analysis more difficult.

While there is a great deal of work attempting to model specific biases (like scatter correction) and remove those effects, there are often residual errors that propagate through to reconstructed images. The effect of bias on the reconstruction is generally dependent on the nature of the residual bias as well as data processing. For example, model-based iterative reconstruction (MBIR) is potentially more dependent on an accurate model than filtered-backprojection (FBP).

To address these problems, the present disclosure, in certain aspects, provides a generalized strategy to estimate unmodeled biases for situations, for example, where the underlying source is either unknown or difficult to estimate directly. To illustrate, in some embodiments, two exemplary cases are considered: 1) a one-dimensional (1D) unmodeled bias which is only a function of projection angle (e.g., 1D cases caused by an unmodeled X-ray tube warm-up effect, where x-ray fluence drifts throughout the scan (common in many CBCT systems that do not have a dedicated detector to estimate barebeam fluence)), and 2) a 2D bias which is an unknown function of both radial detector position or bin and projection angle (e.g., an unknown functional comprised of the weighted sum of Gaussian functions of unknown amplitude and widths). In some embodiments, a convolutional neural network (CNN) framework for CT projection-domain de-biasing is used, which consists of the ResUNet architecture and a spatial-frequency loss function which incorporates intermediate information about the reconstruction. In certain embodiments, this exemplary framework is applied to the two bias scenarios and shows a reduction in reconstruction errors in both cases for both FBP and MBIR.

In some embodiments, developments in machine learning are leveraged to provide other artificial neural network (ANN) based solutions for estimating unknown biases in CT. In some of these embodiments, for example, an ANN is used to estimate unbiased projections from many example biased and unbiased sinogram pairs. This methodology is distinct from the application of machine learning to the estimation of specific biases—e.g. using convolutional neural networks to perform scatter correction of CT data [Maier et al., “Deep Scatter Estimation (DSE): Accurate Real-Time Scatter Estimation for X-Ray CT Using a Deep Convolutional Neural Network,” J Nondestruct Eval 2018; 37(3):57]. These and other features of the present disclosure will be apparent upon complete review of the present disclosure, including the accompanying figures.

### Exemplary Methods

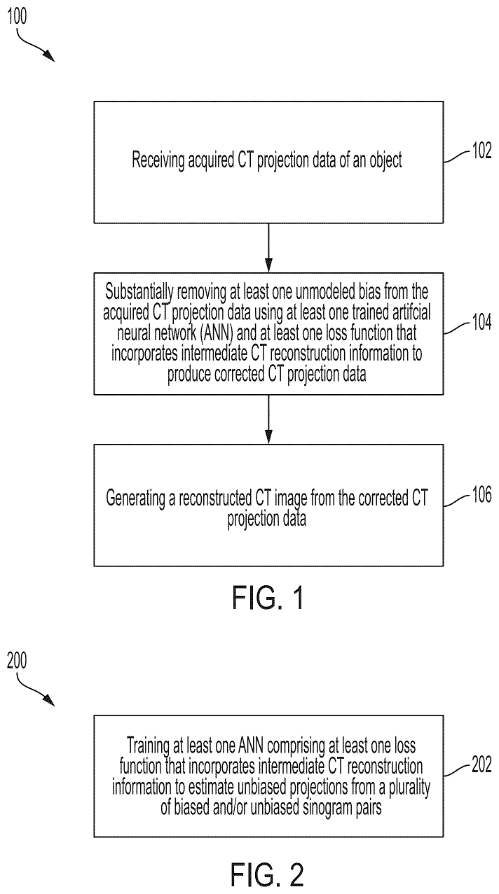

The present disclosure provides various methods of reconstructing computed tomography (CT) images or of generating artificial neural networks (ANNs) to estimate unmodeled bias from acquired CT projection data. To illustrate, FIG. 1 is flow chart that schematically depicts exemplary method steps according to some aspects disclosed herein. As shown, method 100 includes receiving acquired CT projection data of an object (step 102). Method 100 also includes substantially removing at least one unmodeled bias from the acquired CT projection data using at least one trained artificial neural network (ANN) and at least one loss function that incorporates intermediate CT reconstruction information to produce corrected CT projection data (step 104). In addition, method 100 also includes generating a reconstructed CT image from the corrected CT projection data (step 106).

To illustrate, FIG. 2 is flow chart that schematically depicts another exemplary method according to some aspects disclosed herein. As shown, method 200 includes training at least one ANN comprising at least one loss function that incorporates intermediate CT reconstruction information to estimate unbiased projections from a plurality of biased and/or unbiased sinogram pairs to thereby generate an ANN to estimate unmodeled bias from acquired CT projection data (step 202).

In some embodiments, a source of the unmodeled bias is unknown or substantially indeterminable. In some embodiments, the methods include using at least one physical model of a CT data collection process to identify the unmodeled bias from the acquired CT projection data. In some embodiments, the unmodeled bias includes a potential to propagate through a reconstruction process to create one or more artifacts and/or one or more errors in an estimation of attenuation coefficients. In some embodiments, the unmodeled bias is selected from the group consisting of, for example, a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

In some embodiments, the unmodeled bias includes a residual bias. In some of these embodiments, the residual bias is selected from the group consisting of, for example, an inexact knowledge of an x-ray spectrum for beam hardening correction, a drift in one or more x-ray detectors, a tube warm-up effect, and an inexact modeling of scatter. In some embodiments, the unmodeled bias is one-dimensional (1D). In some of these embodiments, the 1D unmodeled bias is a function of projection angle. In some embodiments, the unmodeled bias is two-dimensional (2D). In some of these embodiments, the 2D unmodeled bias is a function of radial detector bin and projection angle. In some embodiments, the unmodeled bias is substantially specific to an anatomy of the object (e.g., a given patient or other subject). In some embodiments, the unmodeled bias comprises scatter and/or differential beam hardening.

In some embodiments, the loss function comprises a spatial-frequency loss function, an image domain loss function, and/or a projection domain loss function. In some embodiments, the loss function comprises a mean squared error between ramp-filtered ANN-corrected and unbiased projection data in a frequency domain. In some embodiments, the spatial-frequency loss function comprises the Formula:

$$Loss = \frac{1}{N} {\sum }_{i} {\sum }_{j} {\{ F [ Ramp filter * {y}_{CNN corrected} ( i , j ) ] - F [ Ramp filter * {y}_{unbiased} ( i , j ) ] \}}^{2}$$

where N is a number of measurements, F{ }represents a Fourier transform, * represents convolution, y_{CNN corrected} represents de-biased projections, y_{unbiased} are true projections, and (i,j) are measurement indices.

In some embodiments, the acquired CT projection data of the object is generated using a single energy CT (SECT) technique. In some embodiments, acquired CT projection data of the object is generated using a spectral CT technique. In some of these embodiments, for example, the spectral CT technique comprises a dual energy CT (DECT) technique and/or a photon counting CT technique.

In some embodiments, the ANN is trained to estimate unbiased projections from a plurality of biased and/or unbiased sinogram pairs (i.e., a plurality of biased sinogram pairs, a plurality of unbiased sinogram pairs, or a plurality of pairs of both biased and unbiased sinograms). In some embodiments, the plurality of biased and/or unbiased sinogram pairs comprises about 10,000, 20,000, 30,000, 40,000, 50,000, 60,000, 70,000, 80,000, 90,000, 100,000, or more biased and/or unbiased sinogram pairs. In some embodiments, the ANN is not trained to estimate a specific bias type. In some embodiments, the ANN comprises a convolutional neural network (CNN) that comprises at least one encoding phase, at least one bridge, at least one decoding phase, and at least one two-dimensional (2D) convolutional layer. In some of these embodiments, the 2D convolutional layer is configured to learn a one-dimensional (1D) bias map. In some of these embodiments, the 2D convolutional layer is configured to learn a 2D bias map and/or a 3D bias map.

In some embodiments, the corrected CT projection data is improved in a projection domain, an image domain, or both a projection domain and an image domain relative to the acquired CT projection data. In some embodiments, the methods include using a reconstruction technique selected from the group consisting of: a model-based iterative reconstruction (MBIR) technique, a deep learning technique, and a filtered-backprojection (FBP) technique. In some embodiments, the methods include receiving the x-ray CT data at one or more x-ray detectors configured and positioned to detect x-ray energy transmitted through the object (e.g., a given patient or other subject) from one or more x-ray energy sources to generate the acquired CT projection data of the object.

### Exemplary Systems and Computer Readable Media

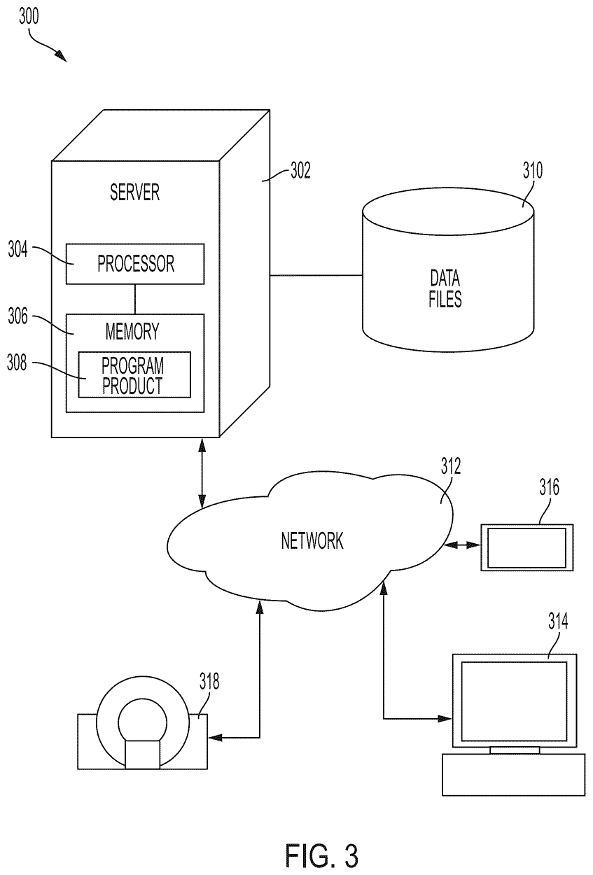

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 300 includes at least one controller or computer, e.g., server 302 (e.g., a search engine server), which includes processor 304 and memory, storage device, or memory component 306, and one or more other communication devices 314 and 316 (e.g., client-side computer terminals, telephones, tablets, laptops, other mobile devices, etc.) positioned remote from and in communication with the remote server 302, through electronic communication network 312, such as the Internet or other internetwork. Communication device 314 typically includes an electronic display (e.g., an internet enabled computer or the like) in communication with, e.g., server 302 computer over network 312 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 300 also includes program product 308 stored on a computer or machine readable medium, such as, for example, one or more of various types of memory, such as memory 306 of server 302, that is readable by the server 302, to facilitate, for example, a guided search application or other executable by one or more other communication devices, such as 314 (schematically shown as a desktop or personal computer). In some aspects, system 300 optionally also includes at least one database server, such as, for example, server 310 associated with an online website having data stored thereon (e.g., acquired CT projection data, etc.) searchable either directly or through search engine server 302. System 300 optionally also includes one or more other servers (e.g., comprising a trained ANN that is used to estimate unmodeled bias from acquired CT projection data) positioned remotely from server 302, each of which are optionally associated with one or more database servers 310 located remotely or located local to each of the other servers. The other servers can beneficially provide service to geographically remote users and enhance geographically distributed operations.

As understood by those of ordinary skill in the art, memory 306 of the server 302 optionally includes volatile and/or nonvolatile memory including, for example, RAM, ROM, and magnetic or optical disks, among others. It is also understood by those of ordinary skill in the art that although illustrated as a single server, the illustrated configuration of server 302 is given only by way of example and that other types of servers or computers configured according to various other methodologies or architectures can also be used. Server 302 shown schematically in FIG. 3, represents a server or server cluster or server farm (e.g., comprising a trained ANN that is used to estimate unmodeled bias from acquired CT projection data) and is not limited to any individual physical server. The server site may be deployed as a server farm or server cluster managed by a server hosting provider. The number of servers and their architecture and configuration may be increased based on usage, demand and capacity requirements for the system 300. As also understood by those of ordinary skill in the art, other user communication devices 314 and 316 in these aspects, for example, can be a laptop, desktop, tablet, personal digital assistant (PDA), cell phone, server, or other types of computers. As known and understood by those of ordinary skill in the art, network 312 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 308 is optionally in the form of microcode, programs, cloud computing format, routines, and/or symbolic languages that provide one or more sets of ordered operations that control the functioning of the hardware and direct its operation. Program product 308, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 308 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 308 is optionally copied from the computer-readable medium to a hard disk or a similar intermediate storage medium. When program product 308, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this disclosure provides systems that include one or more processors, and one or more memory components in communication with the processor. The memory component typically includes one or more instructions that, when executed, cause the processor to provide information that causes at least one reconstructed CT image and/or the like to be displayed (e.g., via communication device 214 or the like) and/or receive information from other system components and/or from a system user (e.g., via communication device 214 or the like).

In some aspects, program product 308 includes non-transitory computer-executable instructions which, when executed by electronic processor 304 perform at least: receiving acquired CT projection data of an object; substantially removing at least one unmodeled bias from the acquired CT projection data using at least one trained artificial neural network (ANN) and at least one loss function that incorporates intermediate CT reconstruction information to produce corrected CT projection data; and generating a reconstructed CT image from the corrected CT projection data.

System 300 also typically includes additional system components (e.g., CT imaging device 318) that are configured to perform various aspects of the methods described herein. In some of these aspects, one or more of these additional system components are positioned remote from and in communication with the remote server 302 through electronic communication network 312, whereas in other aspects, one or more of these additional system components are positioned local, and in communication with server 302 (i.e., in the absence of electronic communication network 312) or directly with, for example, desktop computer 314. Although not within view, CT imaging device 318 includes at least one x-ray energy source and at least one x-ray detector configured and positioned to detect x-ray energy transmitted through an object (e.g., a subject or the like) from the x-ray energy source. System 300 also includes at least one trained artificial neural network (ANN) and at least one loss function that incorporates intermediate CT reconstruction information that are configured to remove unmodeled bias from acquired CT projection data.

### EXAMPLE

### Methods

### A. Specific Bias Scenarios and the Dependence on Data Processing

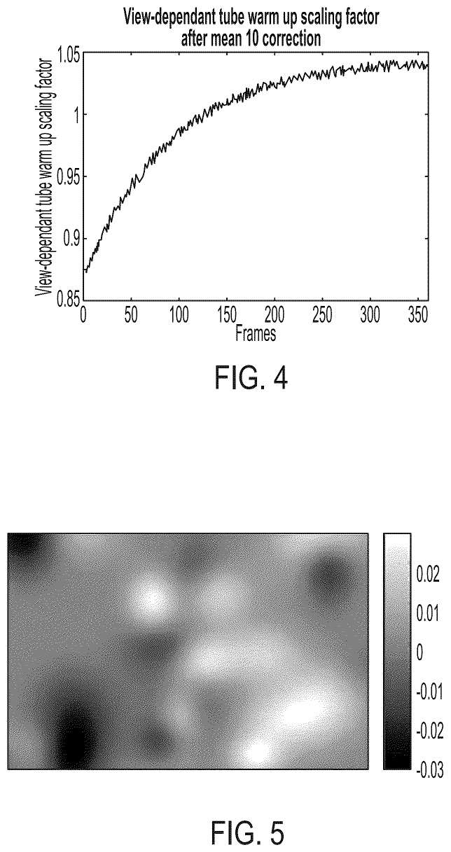

Case 1: 1D bias as a function of projection angle: Based on observations of tube warm-up in a rotating anode radiography/fluoroscopic x-ray source, the following model for tube warm-up was devised:

$$I ( t ) = {I}_{0} \cdot f ( t ) where f ( t ) = K \cdot ( 1 - {e}^{- \frac{t}{τ}} ) + σ \qquad ( 1 )$$

where I₀ is the nominal barebeam fluence, f(t) is the step response of a 1st-order system, K is the steady-state output, τ is the time constant which characterizes how fast the response of the system converges to the steady-state output, and a is the Gaussian noise for adding shot-to-shot randomness to the tube warm up model. This model is used to generate biased data using parameters I₀, K, τ and a randomly chosen from 2.5×10⁴ to 6.5×10⁴, 1 to 1.3, 60 to 200 and 0.001 to 0.01, respectively. Though we have a model for this effect, we will presume it to be unknown, except for a well-characterized mean I₀ over the duration of the scan. All data explored in this scenario is fully truncated to eliminate the “simple” solution of finding an air region in each projection to compensate for the varying fluence. A sample bias realization is shown in FIG. 4.

Case 2: 2D bias as a function of both projection angle and (radial) detector bin: To create a more complicated unknown bias, we modeled a residual error from an inaccurate pre-log correction as a set of 2D Gaussian functions being added to the CT projection data in measurement domain, which is as follows:

$${y}_{biased} = {y}_{unbiased} + {\sum }_{i = 1}^{N} {f}_{i} ( {θ}_{i} , {r}_{i} ) where {f}_{i} ( {θ}_{i} , {r}_{i} ) = {A}_{i} \cdot exp [ - ( \frac{{( θ - {θ}_{i} )}^{2}}{2 {σ}_{θ i}^{2}} + \frac{{( r - {r}_{i} )}^{2}}{2 {σ}_{ri}^{2}} ) ] \qquad ( 2 )$$

where γ represents pre-log data, N is the total number of 2D Gaussian blobs beingadded to each projection, Aᵢ represents the amplitude of the blob, (θᵢ, rᵢ) represents the center of the blob in the sinogram and (σ_{θi}, σᵣᵢ) represents spread of the blob in each direction. The parameters N, A, θᵢ, r and σ_{θi} (σᵣᵢ) are uniformly randomly chosen from 15 to 30, −5% mean measurements to +5% mean measurements, 0 to 256, 0 to 360 and 15 to 30, respectively. A sample bias realization is shown in FIG. 5.

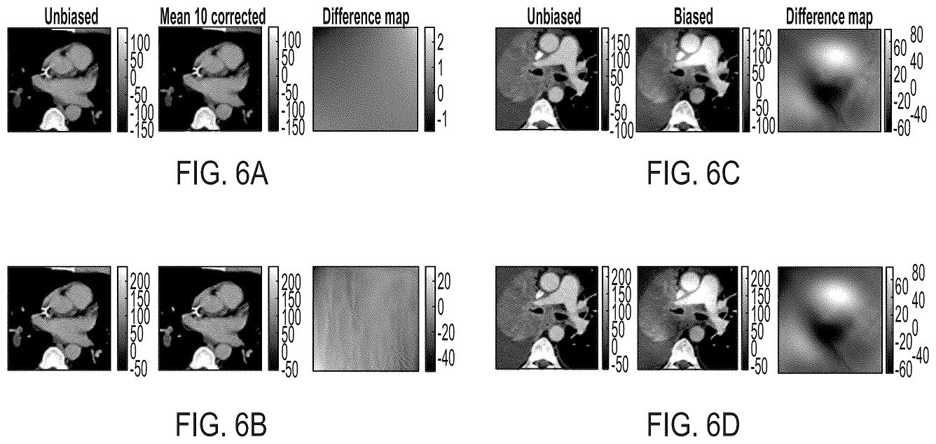

Reconstruction methods: In our investigations, we used two reconstruction methods: 1) Standard FBP using a Hamming apodized ramp filter with 0.8 cutoff; and 2) a specific MBIR approach—quadratic penalized-likelihood (PL) reconstruction using 100 iterations of separable quadratic surrogates, 12 subsets, and a first-order neighborhood penalty with penalty weight of 10⁵. All projection data in this work used a fan-beam geometry with source-to-axis distance (SAD) of 800 mm, source-to-detector distance (SDD) of 1000 mm and square pixels of 0.278 mm to generate noiseless projection data. To illustrate the effects of the two bias scenarios, we conducted a small experiment summarized in FIG. 6 showing an unbiased reconstruction, a reconstruction where only the mean I₀ over the entire scan was known, and the difference image for both FBP and PL. Note that reconstruction bias for the 1D is very small for FBP but increases by a factor of 10 for PL. For the 2D bias, both FBP and PL are affected similarly with a 5% deviation in projection value resulting in noticeable artifacts in both FBP and PL of up to 80 HU. Thus, the impact of bias depends both on the nature of the bias and the data processing approach.

### B. Design of a CNN Debiasing Framework: Architecture and the Loss Function

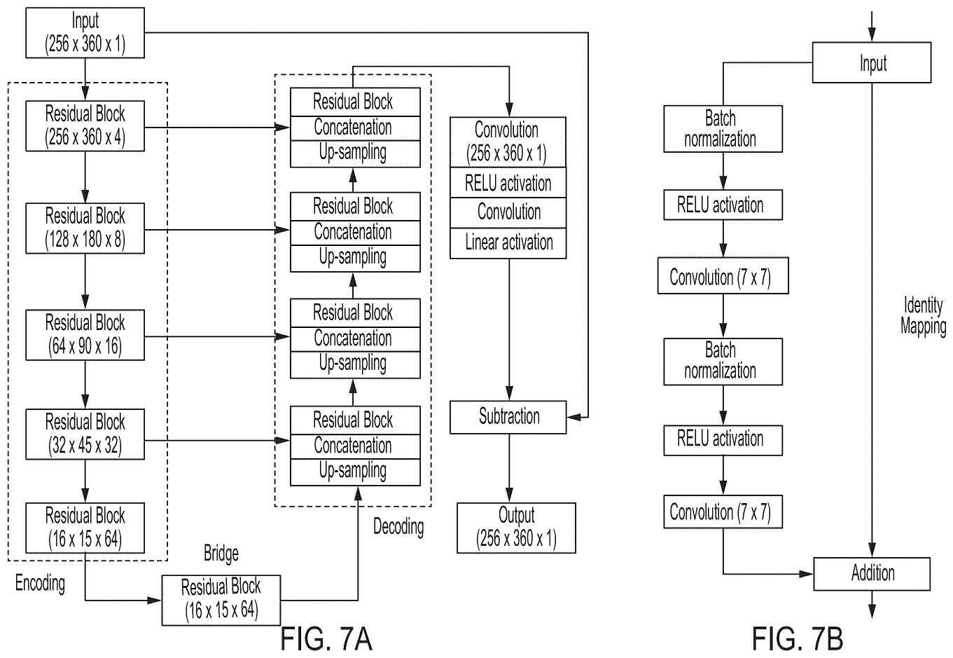

We seek a general machine learning approach to try to estimate the underlying unbiased projections (e.g. learning how to use intrinsic criteria, etc.) from unbiased and biased data pairs. For this effort we adopt a CNN architecture inspired by Deep ResUNet [Zhang et al., “Road Extraction by Deep Residual U-Net,” IEEE Geoscience and Remote Sensing Letters, 2018; 15(5),749-753]. The residual unit helps ease training of the network, and the concatenations between residual units in encoding and decoding phase facilitates information propagation without degradation, which helps preserve structural information of the projection data. Specifically, a 9-level architecture of deep ResUNet has been built, which is illustrated in FIG. 7. The input of the CNN is of size 256×360. The CNN consists of three parts: an encoding phase, bridge, and decoding phase. The encoding phase contains four residual blocks, each with filters of size 7×7 and strides of 2×2 (the last one being 2×3), for encoding the inputs into compact representations of size 16×15. The bridge serves to connect the encoding and decoding phase. For the decoding phase, it also contains four residual blocks with the same settings as in the encoding phase, but with the up-sampling process before each residual block. Finally, there is an additional 2D convolutional layer to learn either a 1-D or 2-D bias map (as appropriate to each bias scenario) which is subtracted it from the input.

The selection of an appropriate loss function requires care. A projection-domain metric is desirable for fast and efficient computation; however, the biases that are important are those that propagate through the reconstruction. Thus, one might prefer an image-domain loss function, but this requires reconstruction as part of the metric. To balance these goals we selected a loss function that weighs the relative importance of projection-domain spatial-frequencies. Specifically, since reconstructions explicitly (FBP) or implicitly (MBIR) perform high-pass filtering of projection data, we choose the mean squared error between ramp-filtered CNN-corrected and unbiased projection data in frequency domain as the loss function:

$$Loss = \frac{1}{N} {\sum }_{i} {\sum }_{j} {\{ F [ Ramp filter * {y}_{CNN corrected} ( i , j ) ] - F [ Ramp filter * {y}_{unbiased} ( i , j ) ] \}}^{2} \qquad ( 3 )$$

The main advantage of this loss function is that it incorporates intermediate information about the CT reconstruction, but maintains relatively low computational cost with a projection-domain metric.

For training we use a database of 45,000 CT axial slices of different patients from the DeepLesion dataset [Yan et al., “DeepLesion: Automated Mining of Large-Scale Lesion Annotations and Universal Lesion Detection with Deep Learning,” Journal of Medical Imaging 5(3), 036501 (2018)]. We divide these slices into a training set of size 40,000, validation set of size 4,000 and test set of size 1,000. These three sets of data have no sharing of patient anatomy information. Inputs to the network for training and evaluation have simple gain correction applied by using the mean bare beam fluence I̅₍̅ₜ̅₎̅ and conversion of both unbiased and biased projection data into log-transformed line integrals. The CNN for 1D and 2D de-biasing were trained for 100 and 50 epochs, respectively, with mini-batch size of 50 and learning rate of 0.0005. Computation time for the training are about 40 and 20 hours with a PC installing single NVIDIA GTX TITAN X GPU and 2.40 GHz Intel (R) Xeon (R) CPU.

### RESULTS

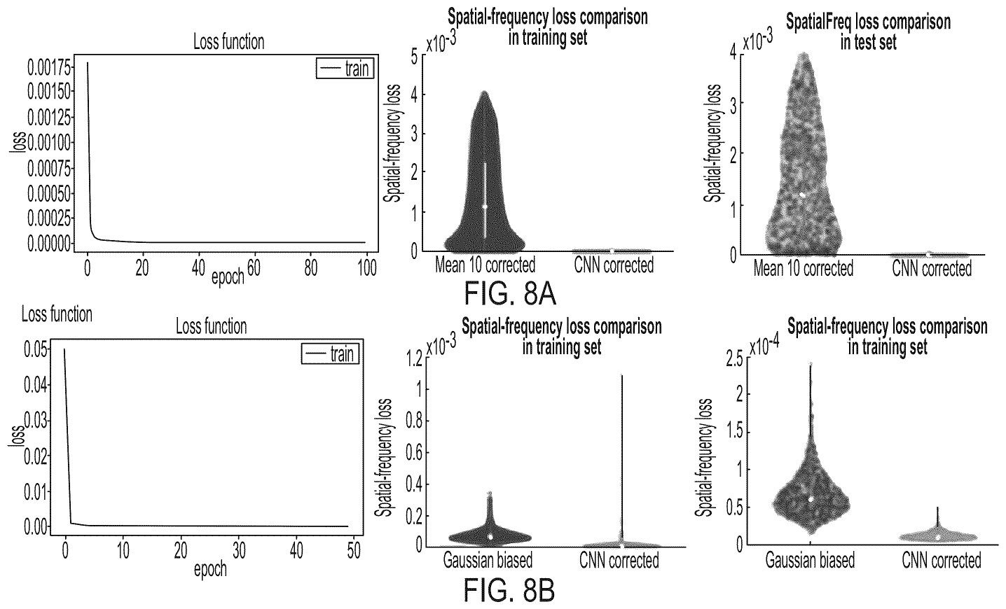

FIG. 8 (panels a and b) show the training history of the CNN for 1D and 2D debiasing. Both plots indicate the convergence of the training process. For 1D debiasing, violin plots are plotted to show and compare the distribution of spatial frequency loss between simple mean-I₀-corrected projection data and CNN-corrected data in both training and test datasets. The mean spatial-frequency losses of mean-I₀-corrected projection data in training and test datasets are both 0.0014, while for the CNN they are significantly reduced to 1.45×10⁻⁶ and 4.65×10⁻⁶, respectively. For 2D debiasing, the mean spatial-frequency losses of 2D biased projection data in training and test datasets are 7.17×10⁻⁵ and 6.70×10⁻⁵, while for CNN they have been reduced to 9.93×10⁻⁶ and 1.12×10⁻⁵, respectively. All the above indicate that the CNN succeeds in training and extends well to unseen data.

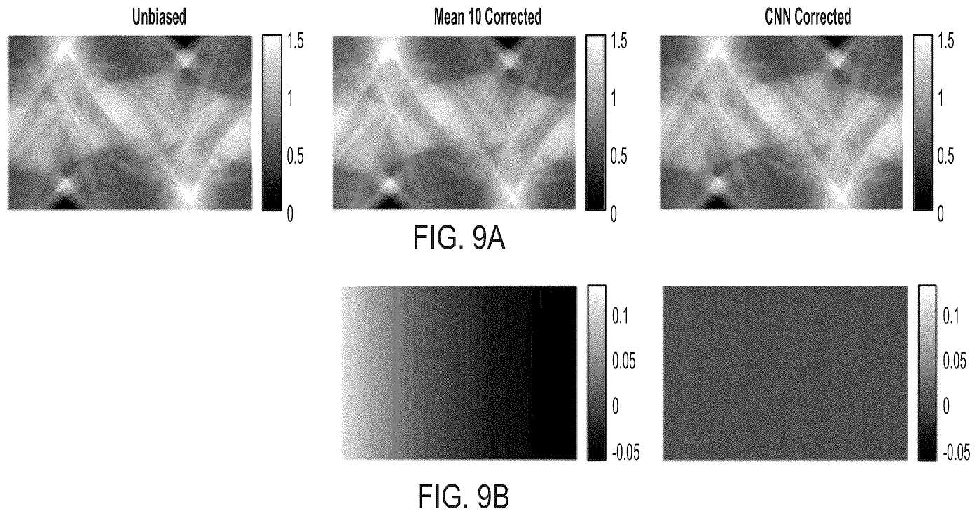

We also evaluated the improvement of CNN corrected projection data in both projection domain and image domain. For 1D debiasing, FIG. 9 shows a representative example where the CNN helps eliminate the view-dependent tube warm up scaling factors in log-scale projection data, with the tube warm up trend function in the difference map between the mean-I₀-corrected and unbiased projection data being flattened close to zero. The mean squared error (MSE) with respect to the unbiased projection data is reduced from 2.10×10⁻³ to 2.48×10⁻⁶—a three-order of magnitude improvement.

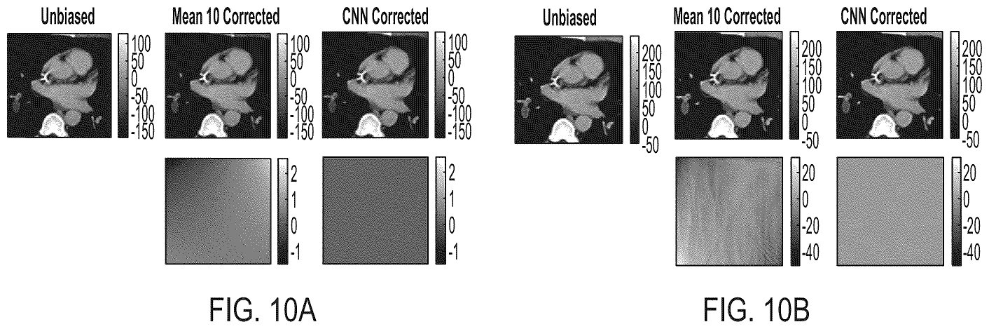

For image-domain improvements we consider FBP and PL reconstructions. Again, a representative sample is shown in FIG. 10 for the 1D bias scenario. The difference maps in FIG. 10a shows that for FBP the 1D bias in projection domain results in a relatively minor skew of HU values across the field. FIG. 10b shows PL reconstruction is significantly more sensitive to the simple mean-I₀-correction with the maximum and minimum errors around 30 HU and −40 HU. Both FBP and PL reconstructions benefit from the CNN corrected projections. The CT number bias is significantly reduced, as can be seen in the difference maps with values close to zero everywhere. The MSE in FBP and PL has been reduced from 0.06 HU² to 3.67×10⁻⁴ HU² and from 145.85 HU² to 0.11 HU², respectively.

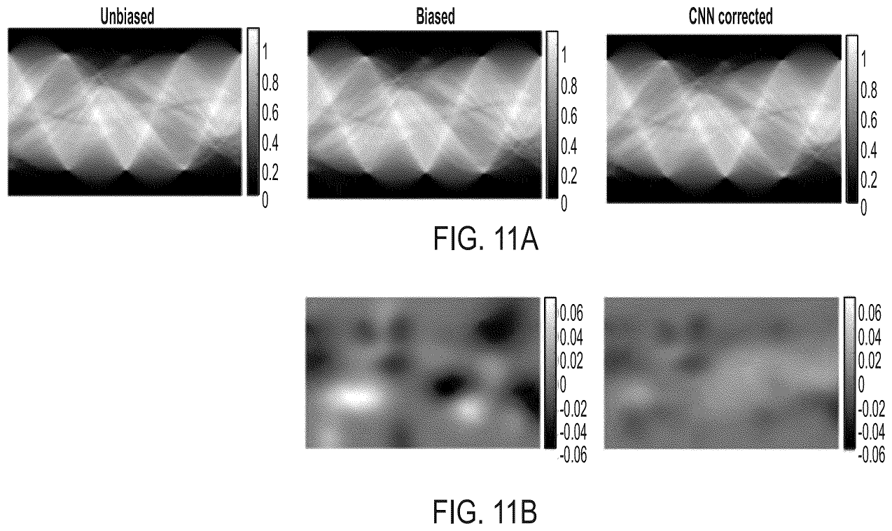

For 2D debiasing, FIG. 11 shows a significant reduction in bias using the CNN. The debiasing is incomplete in this case but much lower as shown by the reduction in intensity of the major bright and dark “blobs” in the difference map. Quantitatively, the MSE with respect to unbiased projection reduces from 3.52×10⁻⁴ to 8.36×10⁻⁵.

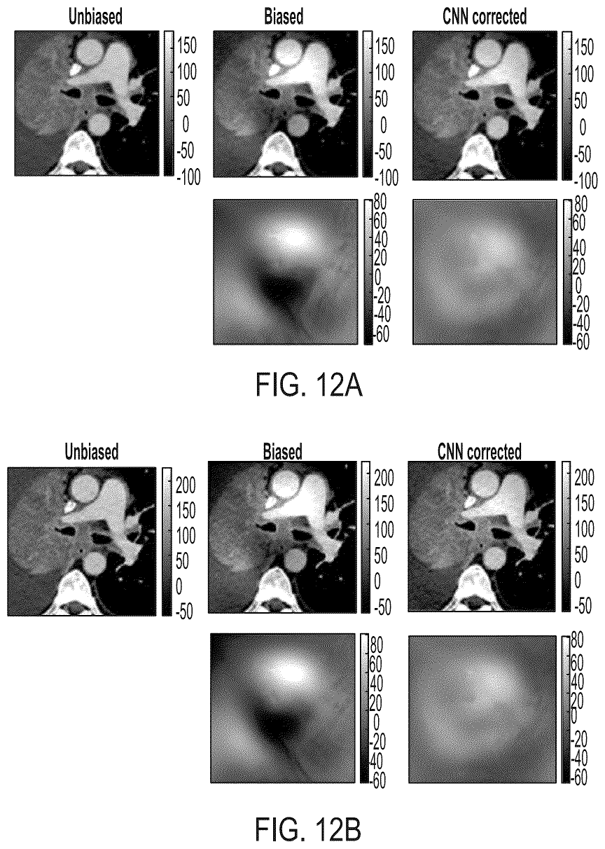

As shown in FIG. 12, the reconstruction of the biased projections causes noticeable and similar artifacts in both FBP and PL reconstructions, evident as the bright and dark regions at the center of the ROI. The maximum and minimum CT number biases are about 80 HU and −60 HU. CNN-corrected projections result in significant reductions of the dark and bright regions with more uniform difference maps. Quantitatively, the MSE in FBP and PL reconstructions have been reduced from 714.56 HU² to 278.35 HU² and from 889.94 HU² to 327.86 HU², respectively.

### CONCLUSIONS and DISCUSSION

In this example we have investigated the impact and a mitigation strategy for unknown biases in CT data. We considered two different classes of bias that might be found in CT projection data representing potential unknowns that vary as a function of projection angle only; or jointly with projection angle and radial detector bin. Different kinds of bias can have significantly different impact on a reconstruction with additional dependencies on the data processing/reconstruction approach. We developed a machine learning approach to exploit intrinsic properties of sinogram data and as well as the data-driven properties of the particular nature of the bias classes and CT datasets under investigation. We found that the ResUNet combined with the spatial-frequency loss function was able to predict these biases allowing for correction and mitigation of the associated artifacts with increased quantitation. This methodology can be applied to physical CT data whose data has unknown bias contamination.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of reconstructing a computed tomography (CT) image, the method comprising:
receiving acquired CT projection data of an object;
substantially removing at least one unmodeled bias from the acquired CT projection data using at least one trained artificial neural network (ANN) and at least one loss function that incorporates intermediate CT reconstruction information to produce corrected CT projection data; and,
generating a reconstructed CT image from the corrected CT projection data, thereby reconstructing the CT image.

2. (canceled)

3. The method of claim 1, wherein a source of the unmodeled bias is unknown or substantially indeterminable.

4. The method of claim 1, wherein the corrected CT projection data is improved in a projection domain, an image domain, or both a projection domain and an image domain relative to the acquired CT projection data.

5. The method of claim 1, comprising using at least one physical model of a CT data collection process to identify the unmodeled bias from the acquired CT projection data.

6. The method of claim 1, comprising using at least one physical model of a CT data collection process, and/or CT data collected from physical calibration phantoms to identify the unmodeled bias from the acquired CT projection data.

7. (canceled)

8. (canceled)

9. The method of claim 1, wherein the unmodeled bias comprises a potential to propagate through a reconstruction process to create one or more artifacts and/or one or more errors in an estimation of attenuation coefficients.

10.-13. (canceled)

14. The method of claim 1, wherein the unmodeled bias is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

15. (canceled)

16. The method of claim 1, wherein the ANN is trained to estimate unbiased projections from a plurality of biased and/or unbiased sinogram pairs.

17.-21. (canceled)

22. The method of claim 1, wherein the unmodeled bias comprises at least one residual bias.

23.-27. (canceled)

28. The method of claim 1, wherein the unmodeled bias is substantially specific to an anatomy of the object.

29.-31. (canceled)

32. A computed tomography (CT) system, comprising:
at least one x-ray energy source;
at least one x-ray detector configured and positioned to detect x-ray energy transmitted through an object from the x-ray energy source;
at least one trained artificial neural network (ANN) and at least one loss function that incorporates intermediate CT reconstruction information that are configured to remove unmodeled bias from acquired CT projection data; and,
at least one controller that is operably connected, or connectable, at least to the x-ray detector and to the trained ANN, wherein the controller comprises, or is capable of accessing, computer readable media comprising non-transitory computer executable instructions which, when executed by at least one electronic processor, perform at least:
receiving acquired CT projection data of an object;
substantially removing at least one unmodeled bias from the acquired CT projection data using the trained ANN and the loss function to produce corrected CT projection data; and,
generating a reconstructed CT image from the corrected CT projection data.

33. (canceled)

34. The system of claim 32, wherein a source of the unmodeled bias is unknown or substantially indeterminable.

35. The system of claim 32, wherein the corrected CT projection data is improved in a projection domain, an image domain, or both a projection domain and an image domain relative to the acquired CT projection data.

36. The system of claim 32, wherein the instructions further perform at least:
using at least one physical model of a CT data collection process to identify the unmodeled bias from the acquired CT projection data.

37. (canceled)

38. (canceled)

39. The system of claim 32, wherein the unmodeled bias comprises a potential to propagate through a reconstruction process to create one or more artifacts and/or one or more errors in an estimation of attenuation coefficients.

40.-43. (canceled)

44. The system of claim 32, wherein the unmodeled bias is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

45. The system of claim 32, wherein the ANN is trained to estimate unbiased projections from a plurality of biased and/or unbiased sinogram pairs.

46.-50. (canceled)

51. The system of claim 32, wherein the unmodeled bias comprises at least one residual bias.

52.-56. (canceled)

57. The system of claim 32, wherein the unmodeled bias is substantially specific to an anatomy of the object.

58. (canceled)

59. (canceled)

60. A computer readable media comprising non-transitory computer executable instruction which, when executed by at least electronic processor, perform at least:
receiving acquired CT projection data of an object;
substantially removing at least one unmodeled bias from the acquired CT projection data using at least one trained artificial neural network (ANN) and at least one loss function that incorporates intermediate CT reconstruction information to produce corrected CT projection data; and,
generating a reconstructed CT image from the corrected CT projection data.
